(19) United States
(12) Patent Application Publication
(10) Pub. No.: US 2023/0268865 A1
(43) Pub. Date: Aug. 24, 2023

(54) SYSTEM AND METHOD TO DETECT DRIVE BLOCKAGE IN A DRUG DELIVERY DEVICE

(71) Applicant: Ypsomed AG, Burgdorf (CH)

(72) Inventors: Krista Kappeler, Bern (CH); Bernhard Rytz, Zollbrück (CH)

(21) Appl. No.: 18/310,309

(22) Filed: May 1, 2023

Related U.S. Application Data
(63) Continuation of application No. PCT/EP2021/079911, filed on Oct. 28, 2021.

(30) Foreign Application Priority Data
Mar. 11, 2020 (EP) 20205379.9

(51) Int. Cl. H02P 29/024; A61M 5/142; A61M 5/168

(52) U.S. Cl. CPC H02P 29/024; A61M 5/14248; A61M 5/16831

(57) ABSTRACT

A system and a method detects blockage of an actuating assembly in a drug delivery device with a brushless DC or stepper motor. The Back EMF voltage is periodically measured at the driving contacts of the motor. A blockage indication value is periodically calculated based on the divergence of voltages measured at different driving contacts, and an alarming and/or mitigation action is initiated if the blockage indication value meets a predefined criterion such as exceeding a minimum or maximum value.

### CROSS-REFERENCE TO RELATED APPLICATIONS

This application claims priority to International Application No. PCT/EP2021/079911 filed on Oct. 28, 2021, entitled “SYSTEM AND METHOD TO DETECT DRIVE BLOCKAGE IN A DRUG DELIVERY DEVICE,” which in turn claims priority to European Application No. 20205379.9 filed on Nov. 3, 2020, entitled “SYSTEM AND METHOD TO DETECT DRIVE BLOCKAGE IN A DRUG DELIVERY DEVICE,” each of which is incorporated by reference herein, in their entirety and for all purposes.

### TECHNICAL FIELD

The present invention relates to a drug delivery device with a brushless electrical motor and an actuating assembly, to a method to detect blockage of the actuating assembly, and to a system to provide blockage detection in such a drug delivery device.

### BACKGROUND

A variety of diseases exist that require regular treatment by subcutaneous administration of a medicament, and a number of drug delivery devices have been developed to support a patient in accurately and controllably delivering an amount of drug in a self-administration process. Drug delivery devices include injection devices that are removed from the site of application after each medication event or drug delivery process, as well as infusion devices with a cannula or needle that remains in the skin of the patient for a prolonged period of time. By way of example, diabetes may be treated by administration of insulin by the patients themselves with the help of multivariable-dose insulin injection pens or infusion pumps. Alternatively, patch injectors, wearable injectors or wearable pumps are patched or adhered to the skin of the patient.

Common to all devices for subcutaneous drug delivery is a reservoir to store the fluid medicament, and a fluid path to bring the drug out of the device and into the subcutaneous tissue of a patient. In a majority of injecting or infusion devices the reservoir has a plunger that is mechanically advanced by an actuation assembly - in this case usually a plunger rod - to drive the fluid out of the reservoir into the fluid path and towards the patient. Alternatively, a pump mechanism such as a peristaltic, membrane or piston pump may be used to transport the fluid and effectuate the drug delivery, with a motor-driven actuation assembly generating the mechanical movement.

For drug delivery devices, blockage of the fluid delivery is a major problem which can occur anywhere in the fluid path, e.g. by crystallization of particles in the drug. Such a blockage in the fluid path is also known as occlusion. For safe and reliable drug delivery, detection of blockages and occlusions are an essential requirement.

In many drug delivery devices, an electric motor is used as a driving means. This also means that some electronic circuitry is integrated to control the motion of the motor and consequently the drug delivery. Every blockage in the fluid path, within the reservoir or within the actuating assembly results in a change of torque or driving force at the output of the motor. Therefore, to minimize complexity and cost of such a system, control and supervision of the drug delivery is often realized directly at the motor, for example by analyzing the supply current drawn by motor coils, to avoid extra cost for additional sensors such as optical sensors to supervise the motion of the rotor or pressure sensors to detect excess force in a stall condition.

EP 0341364 B1 describes a drug delivery device with a plunger moving assembly supervision based on the motor current. The motor current is measured during operation of the pump and integrated over a certain time to eliminate spikes. A blockage is reported if the integrated motor current is higher than a pre-defined threshold.

An alternative concept for motor control is known as “back electro-motive force” (Back EMF or BEMF). Sensors are placed at the contacts of an electric motor to sense the voltage induced by motor coils passing the sensor if the motor is rotating. By monitoring these voltages, the motion of the motor can be detected and also used to detect blockage of the delivery. This technique is of special interest if the motor is a stepping motor or a brushless electric (BLDC) motor, where the same coils can be used for driving the motor and for sensing the back EMF voltage signal.

US 2003/0117100 A1 describes a motor control system for a stepping motor using EMF supervision and blockage detection.

US 9,509,243 B2 describes a linear actuation system with back EMF detection of lost steps.

All efforts to supervise the drug delivery and detect blockage back at the driving motor highly depend on the mechanical properties of the actuating assembly or plunger moving assembly. In a system with a rigid and play-free plunger moving assembly any fault along the drive train and the fluid path can immediately be detected at the motor using one of the known techniques. However, with increasing elasticity of the actuating assembly, fault detection such as detection of blockage and occlusion becomes unreliable or even impossible within the accuracy requirements of a drug delivery device. More specifically, in case of an occlusion in the example of a system with a non-rigid plunger moving assembly, the Back EMF voltage signal will not immediately show zero current (complete blockage), because the driving motor will need some time to compress the elasticity of the plunger moving assembly and suppress the rotor movement sufficiently to detect the fault.

WO 2017/017557 A1 describes a method to compensate mechanical play using Back EMF voltage analysis in an actuation system with an elastic element.

To achieve the accuracy of movement and/or fault detection suitable for a drug delivery device, additional sensors and/or mechanisms in the fluid path may be needed, increasing complexity and cost of the drug delivery system.

At the same time it may be advantageous for the mechanical design to allow a controlled amount of elasticity in the plunger moving assembly rather than eliminate the elasticity to avoid the problems described above. The classical linear plunger rod may be replaced by a different mechanism, such as a curved chain of short sticks, a train of balls in a bearing or even a spring, opening up a wide range of new possibilities for the design of drug delivery devices which may be more compact, more ergonomic in outer shape or more cost efficient in manufacturing or use.

There is clearly a need for a system and a method to detect blockage and occlusion in a drug delivery device with a potentially elastic plunger moving assembly, where the blockage is detected directly at the driving motor and no extra sensor is introduced in the reservoir or fluid path.

### SUMMARY

It is an objective of the invention to provide an improved system and method to detect blockage in a medical fluid delivery device. The improvement explicitly includes providing reliable detection of a blockage in a drug delivery device with a non-rigid actuating assembly. A typical application of the improved blockage detection may for example be in a compact, body-worn drug delivery device with a reservoir and a plunger, where relaxed requirements for rigidity of the plunger moving assembly are most advantageous.

This objective is achieved by a drug delivery device with an electric stepper motor or brushless DC motor (31) operatively connected to the actuating assembly and including at least one driving phase, for example two driving phases, each driving phase having a coil (40), a positive driving contact (41), and a negative driving contact (42) to drive the motor. The drug delivery device further includes a voltage measurement circuitry (55) to measure the voltage at or between the driving contacts (41). The drug delivery device further includes an electronic control unit (20) with a control circuitry (50), an electrical power source, a microprocessor, storage and software configured to control the commutation of the motor (31), to supervise the actuation of the drug delivery device and to initiate an alarming and/or mitigation action if a blockage of the actuating assembly (30) has been detected. This detection is realized with a novel method (160) of evaluating the Back EMF voltage signal (60) of the motor which includes the following steps:

Step 1: commutating the motor (31) by applying a pattern of positive driving voltages (61) to the driving contacts (41), such as repeating a commutation cycle with a succession of all positive driving contacts (41) and all negative driving contacts (42).

Step 2: pausing the driving of at least one driving phase periodically, for example twice per commutation cycle, such as pausing between driving the positive and driving the negative driving contact (42) of the same driving phase, to open a recurring measuring window (63) during which the driving contacts (41) of the paused driving phase are controlled by the voltage measuring circuitry (55) to measure a Back EMF voltage signal (60) at or between said driving contacts (41).

Step 3: measuring or sampling, for a sequence of measuring windows (63), the voltage at or between driving contacts (41) of a driving phase and establishing, for each of the sequence of measurement windows (63), a convergence value (80) representing the Back EMF voltage during said measuring window (63).

Step 4: Periodically calculating, for example once for every commutation cycle k, a blockage indication value BI based on the divergence of convergence values CV measured at different driving contacts. Any mathematical expression may be used to assess said divergence, for example a quantification of maxima and minima, as in the formula BI(k) = max (CP(k) -CN(k)), where CP is a convergence value assigned to a positive driving contact (41), CN is a convergence value assigned to a negative driving contact (42). For the purpose of formulating the calculation of BI, an assigning step may further be included, which includes assigning each convergence value (80) to a positive or to a negative driving contact, such as to the positive or to the negative driving contact of the driving phase paused to perform the voltage measurement, for example assigning the convergence value to the driving contact to which the driving pattern has last been applied before starting the measurement window the convergence value is representing; storing the convergence values in sequences per driving contact, such as with driving phases A and B the sequences CP_{A}, CP_{B} for convergence values at the positive driving contacts P_{A}, P_{B} and the sequences CN_{A}, CN_{B} for convergence values at the negative driving contacts N_{A}, N_{B}.

Step 5: deciding that a blockage of the actuation assembly is present if the blockage indication value BI meets a predefined criterion, for example if the blockage indication value BI exceeds a blockage threshold ±MaxConvDiff for a specified minimum number of commutation cycles k within a specified minimum blockage duration; and

Step 6: initiating an alarm and/or a mitigation action if a blockage has been determined.

In the drug delivery device outlined above, two main aspects of the present disclosure are evident: a method (160) using steps 1 to 6 to detect blockage of the actuation assembly, and a system built into the medical device to implement said method (160). More details about both aspects are specified further below.

### BRIEF DESCRIPTION OF THE DRAWINGS

The subject matter of the disclosure will be explained in more detail in the following text with reference to exemplary embodiments which are illustrated in the attached drawings, in which:

FIG. 1 depicts a patch injector according to the present disclosure;

FIG. 2 depicts the patch injector partially opened to show the electronic control unit;

FIG. 3 depicts a selection of the interior of the patch injector with the motor and the actuation assembly;

FIG. 4a depicts an exploded physical view of a brushless stepper motor with two phases;

FIG. 4b depicts the physical arrangement of magnetic poles in the motor;

FIG. 4c depicts a schematic illustration of the magnetic structure of the motor;

FIG. 5a depicts the schematic diagram of a unipolar single phase motor drive circuitry;

FIG. 5b depicts the schematic diagram of a bipolar dual phase motor drive circuitry;

FIG. 6a depicts a driving voltage pattern used to commute the motor with a Back EMF measuring window;

FIG. 6b depicts a commutation cycle with measuring windows;

FIG. 7 depicts a detailed view of the Back EMF voltage signal during a measuring window;

FIG. 8 depicts a set of convergence data used to determine a convergence value;

FIG. 9a depicts a set of sequences of convergence values for two phases;

FIG. 9b depicts a set of sequences of blockage indication valuescalculated as differences of convergence values;

FIG. 10a depicts a set of sequences of smoothed convergence values, differences and mean values for phase A;

FIG. 10b depicts a set of sequences of smoothed convergence values, differences and mean values for phase B;

FIG. 11 illustrates the normalization of convergence values;

FIG. 12a depicts a sequence of normalized smoothed convergence values, and their difference, for phase A;

FIG. 12b depicts a sequence of normalized smoothed convergence values, and their difference, for phase B;

FIG. 13a depicts a sequence of blockage indication values based on a maximum difference of smoothed convergence values in individual phases;

FIG. 13b depicts a sequence of blockage indication values based on a maximum difference of smoothed and normalized convergence values in individual phases;

FIG. 14a depicts a sequence of blockage indication values based on a maximum difference of smoothed convergence values in a combination of multiple phases;

FIG. 14b depicts a sequence of blockage indication values based on a maximum difference of normalized convergence values in a combination of multiple phases;

FIG. 15 depicts a sequence of blockage indication values based on a maximum square error of convergence values; and

FIG. 16 shows a flowchart of steps 1 to 6 of the method for detecting blockage according to the present disclosure.

The reference symbols used in the drawings, and their primary meanings, are listed in summary form in the list of designations. In principle, identical parts are provided with the same reference symbols in the figures.

### DETAILED DESCRIPTION OF THE DISCLOSURE

In the present context, the terms “substance”, “drug”, “medicament” and “medication” are to be understood to include any flowable medical formulation suitable for controlled administration through a means such as, for example, a cannula or a hollow needle, and includes a liquid, a solution, a gel or a fine suspension containing one or more medical active ingredients. A medicament can be a composition including a single active ingredient or a pre-mixed or co-formulated composition with more than one active ingredient present in a single container. Medication includes drugs such as peptides (e.g., insulin, insulin-containing drugs, GLP-1 containing drugs or derived or analogous preparations), proteins and hormones, active ingredients derived from, or harvested by, biological sources, active ingredients based on hormones or genes, nutritional formulations, enzymes and other substances in both solid (suspended) or liquid form but also polysaccharides, vaccines, DNA, RNA, oligonucleotides, antibodies or parts of antibodies but also appropriate basic, auxiliary and carrier substances

The term,,distal” is meant to refer to the direction or the end of the drug delivery device carrying an injection needle or an injection cannula, whereas the term “proximal” is meant to refer to the opposite direction or end pointing away from the needle or cannula.

The term “injection system” or “injector” refers to a device that is removed from the injection site after each medication event or drug delivery process, whereas the term “infusion system” refers to a device with a cannula or needle that remains in the skin of the patient for a prolonged period of time, for example, several hours.

At the core of the present disclosure is a system and a method to detect blockage of an actuating assembly in a medical device with a brushless electric motor by means of a novel way of evaluating Back EMF voltage signals. While blockage detection is a typical system requirement in medical devices, the method of this disclosure may be used for any kind of medical device, with any kind of brushless electric motor and any kind of actuating assembly. In this document, the implementation of disclosure is described in an embodiment of a drug delivery device, more specifically in the example of a wearable patch injector for subcutaneous application of a fluid drug. Any person skilled in the art will have no difficulty applying the same disclosure to the design of a patch pump, mobile pump, stationary pump or any other medical device with an actuating assembly suitable for supervision by analysis of Back EMF voltage signals.

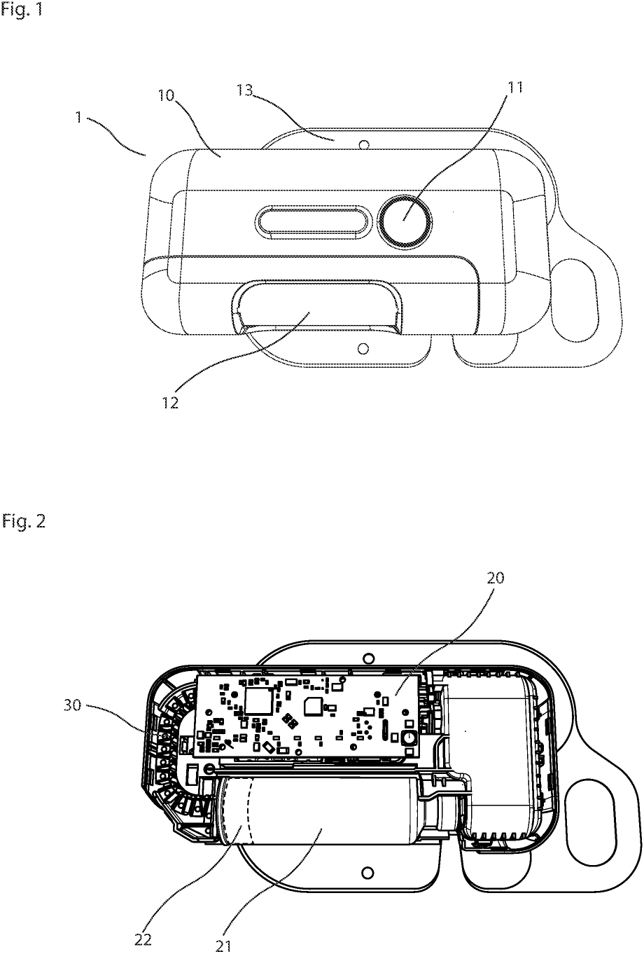

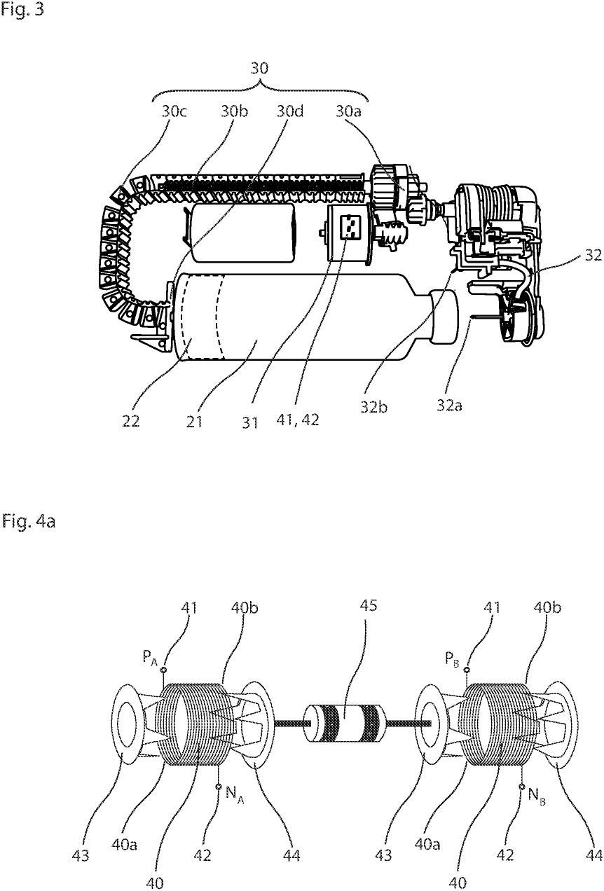

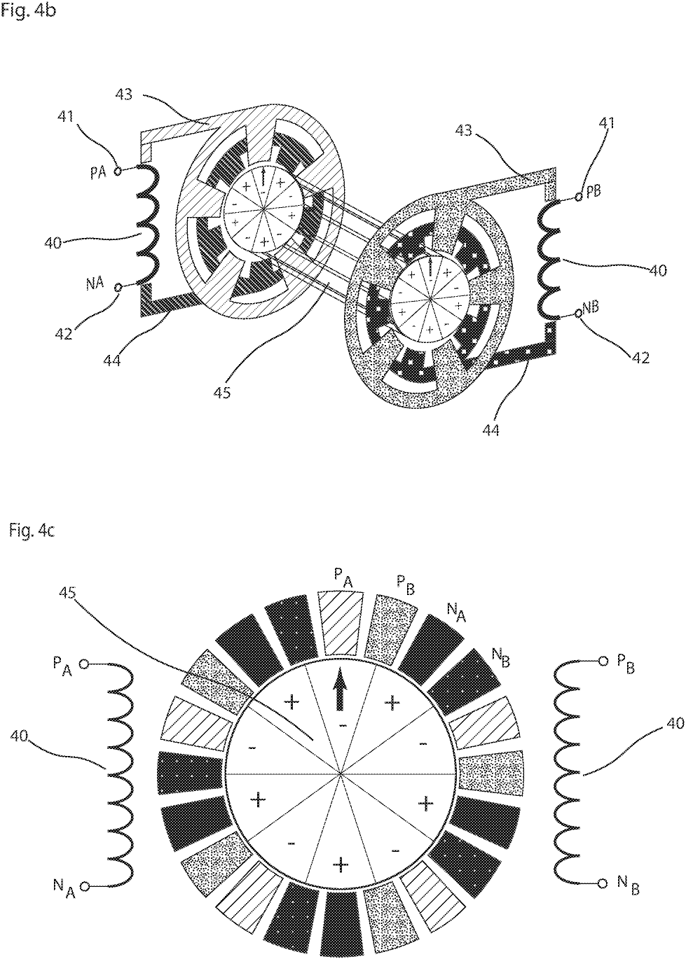

FIG. 1, FIG. 2 and FIG. 3 illustrate the patch injector (1) and the main components thereof. The patch injector (1) has a housing (10) to protect the device from the environment and provide a compact, wearable shape suitable for constant use over several minutes. An adhesive patch assembly (13) is permanently attached to the housing and used to attach the pump to the body of a patient for application. The housing holds a reservoir (21) to contain a medical fluid, a plunger (22) movably disposed in the reservoir (21), and a fluid transport assembly (32) to connect the reservoir to the patient. In this example, the reservoir has a septum and the fluid transport assembly (32) has a needle at the input (32a) to pierce said septum and connect the fluid transport assembly with the reservoir (21). At the output (32b) of the fluid transport assembly (32) is a cannula, configured to be temporarily inserted into the body of the patient for subcutaneous drug delivery. The drug delivery is effectuated by means of a brushless stepper motor (31) commutated via electric driving contacts (41, 42), with a rotor (45) operatively connected to an actuating assembly (30, see FIGS. 3 and 4a). The actuating assembly (30) includes all components necessary to translate the movement of the rotor (45) to an advancement of the medical fluid. In such an embodiment, the actuating assembly (30) is a plunger moving assembly including a gear box assembly (30a), a threaded rod (30b), a segmented rod (30c) and a plunger moving head (30d) mechanically connecting to the plunger (22) and moving the plunger (22) in the reservoir (21) forward. An electronic control unit with an electronic control circuitry (50), an electrical power source (not shown), hardware for electronic and/or visual and/or acoustic communication, a microprocessor, storage and software is packed into the housing and configured to control all system functions such as interacting with a user or interacting with another device involved in the drug delivery therapy, commutating the motor (31), supervising the actuation of the plunger and initiating an alarming action and/or a mitigation action if a blockage of the actuating assembly (30) has been detected. On the outside of the housing (10), a command button (11) is connected to the electronic control unit (20) to initiate drug delivery. A reservoir window (12) in the housing (10) may allow the patient to visually supervise the drug delivery by double-checking the position of the plunger (22) in the reservoir (21).

FIG. 4 illustrates the brushless stepper motor used in an embodiment of disclosure. The motor has two driving phases A and B with two coils (40) radially arranged around the rotor (45), axially next to each other as shown in the exploded view of FIG. 4a. At both ends of each coil, a magnetic flux conductor (43, 44) collects the magnetic field induced by the coil when activated by applying an electric voltage to the driving contacts (41, 42) and distributes the magnetic flux to a number of protrusions, in the described embodiment five protrusions per magnetic flux conductor. The protrusions of the magnetic flux conductors (43, 44) are arranged inside the coil (40) alternately from both ends of the coil (40) to form a number of magnetic pole pairs (see FIG. 4b) used to drive the rotor (45), in the described embodiment five pole pairs per coil (45). The flux conductors of the two coils are arranged in a fixed position axially aligned around the axis of the rotor (45) and rotated by half the angle between two pole pairs, in the example given by 18 degrees. This arrangement forms a stator with two coils (40) and ten magnetic pole pairs as illustrated in the schematic view of FIG. 4c. For the purpose of describing the invention as disclosed, we call one end of a coil (40) the positive end (40a) and the other end of the coil the negative end (40b). This definition is arbitrary, does not reflect any magnetic or electric polarity and could also be swapped between the two ends. Using this definition, each coil (40) has a positive driving contact P (41) and a positive magnetic flux conductor (44) at the positive end (40a), and a negative driving contact N (42) and a negative magnetic flux conductor (45) at the negative end (40b). In the example of the embodiment used for this disclosure, the driving phases A and B with coils A and B (40) have two positive driving contacts P_{A}, P_{B} (41) and two negative driving contacts N_{A}, N_{B} (42). By cyclically applying a positive voltage to a sequence of positive and negative driving contacts, the rotor is rotated or commutated continuously - in the case of a brushless DC motor -or in a number of discrete motor steps per rotation, in the example 20 motor steps per rotation. To emphasize the distinction from micro-steps, motor steps induced by one of such a cyclic change of commutation states are termed “motor full-steps”. Alternative implementations of disclosure may have a motor with a different number of driving phases, such as single phase or triple phase, may have phases with a different number of coils, such as two coils in series or multiple coils in parallel, may have more than two driving contacts per driving phase, such as a middle contact M_{A} between two coils connected in series.

The first three steps of blockage detection according to the invention as disclosed are tightly connected and will be described together in the following paragraphs. The steps are
1) to induce a movement of the rotor by applying a cyclic commutation at the driving contacts
2) periodically pause said commutation and open a measuring window
3) periodically sample the Back EMF voltage signal during said measuring window and establish a convergence value.

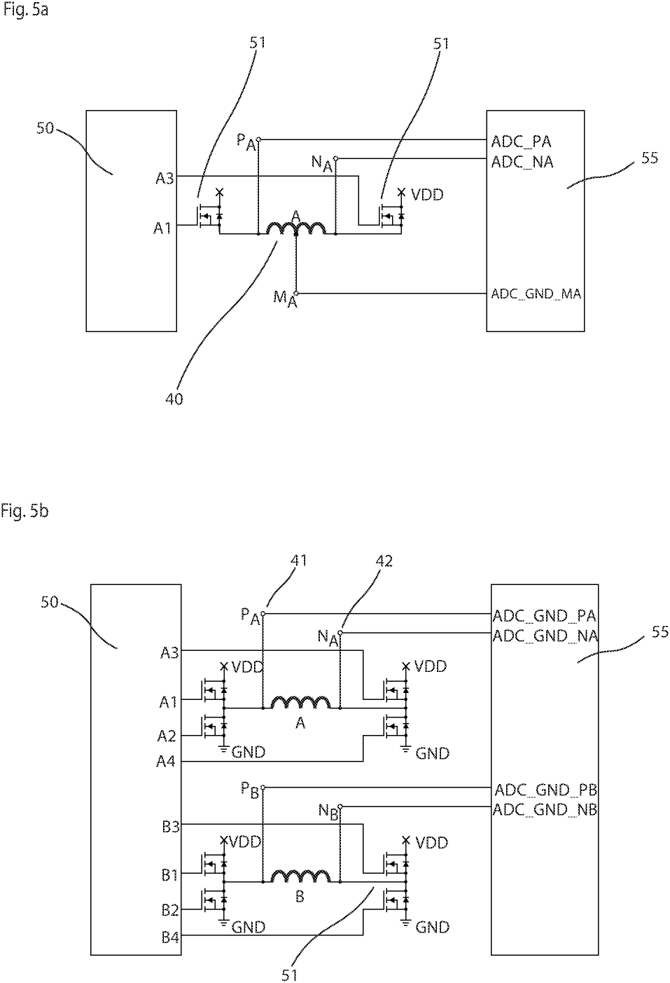

Electronic circuitry to commutate a brushless DC (BLDC) or stepper motor is well established in prior art. FIG. 5a shows a schematic diagram of a unipolar drive circuitry for a single phase motor, where the positive driving contact P_{A} and the negative driving contact N_{A} are connected to a positive driving voltage VDD via MOS-FET switches (51). The control circuitry (50) is configured to operate the switches and apply the driving voltage VDD alternatingly to P_{A} and N_{A} while the middle driving contact M_{A} remains connected to the electrical ground GND. In the example of FIG. 5a, a voltage measuring circuitry is connected to all three driving contacts P_{A}, N_{A} and M_{A}. This allows the control circuitry to pause the commutation and use the voltage measurement circuitry to sample the course of voltage at the driving contacts and determine the voltage induced by back electro-motive force (Back EMF). Voltage sampling is typically performed by repeatedly and synchronously triggering a number of analog-digital converters (ADC) in the measurement circuitry. The Back EMF voltages may simply be read from an ADC at a particular driving contact or calculated from a multitude of voltages sampled at a multitude of driving contacts of the same driving phase, for example by subtracting the voltage at N_{A} from the voltage at P_{A}. The measurement circuitry may connect select driving contacts to VDD or to GND to control the sampling of Back EMF voltages. Instead of Back EMF voltages, Back EMF currents may be sampled in the same way. In both cases sequences of sampled values are analyzed, for example once for each measuring window, and processed to obtain one convergence value per measuring window (80, FIG. 8), representative of the Back EMF signal during said measuring window.

In FIG. 5b, the driving and measuring circuitry is extended to an embodiment with a two-phase motor and a bipolar commutation. Each driving phase A, B has a coil (40) and four MOS-FET switches (51) forming an H-bridge. The control circuitry (50) is configured to operate the switches (51) and to apply a positive driving voltage cyclically to all positive and negative driving contacts, for example by repeating the sequence P_{A}, P_{B}, N_{A}, N_{B}. The sequence of driving contacts may vary with the design of the motor and with the direction of rotation. Whenever a driving contact is connected to VDD, the drive contact at the opposite end of the same coil is connected to the electrical ground GND. In this embodiment, as there are four driving contacts, the sequence includes four motor full-steps (62) until the sequence is repeated, defining a commutation cycle of four motor full-steps (62). The direction of rotation can also be controlled by choosing the sequence of driving contacts in a commutation cycle according to the design of the motor. Just like with the unipolar operation, pauses in commutation allow the voltage measurement circuitry in FIG. 5b to connect the driving contacts (41, 42) to VDD or GND, to sample the voltages at the driving contacts, and to obtain one convergence value (80, FIG. 8) per measuring window (63), representative of the Back EMF signal during said measuring window.

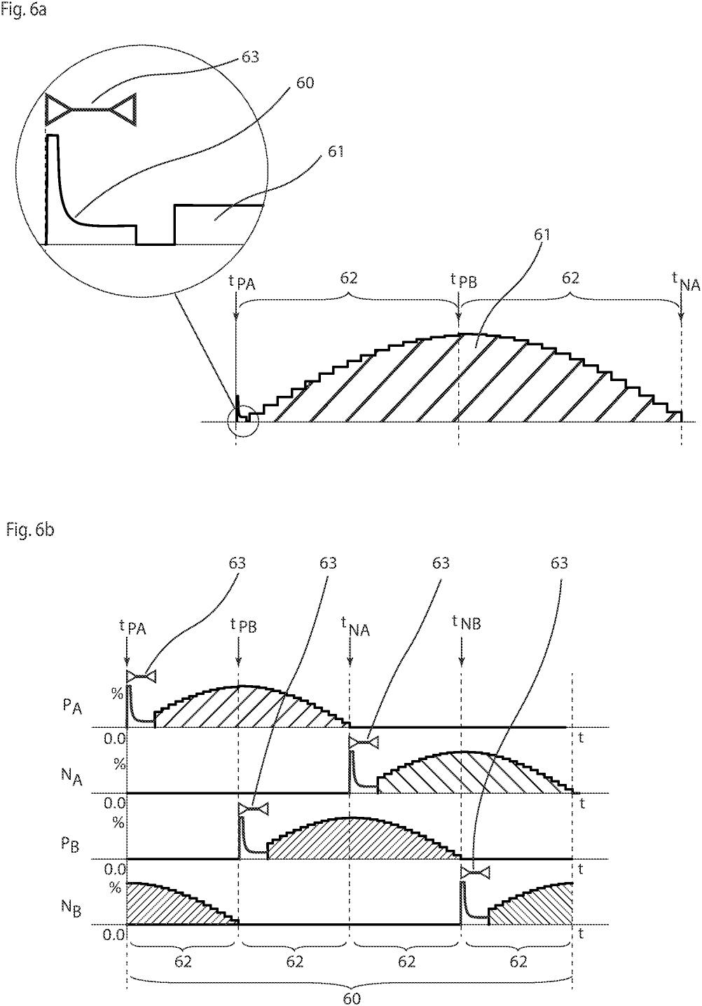

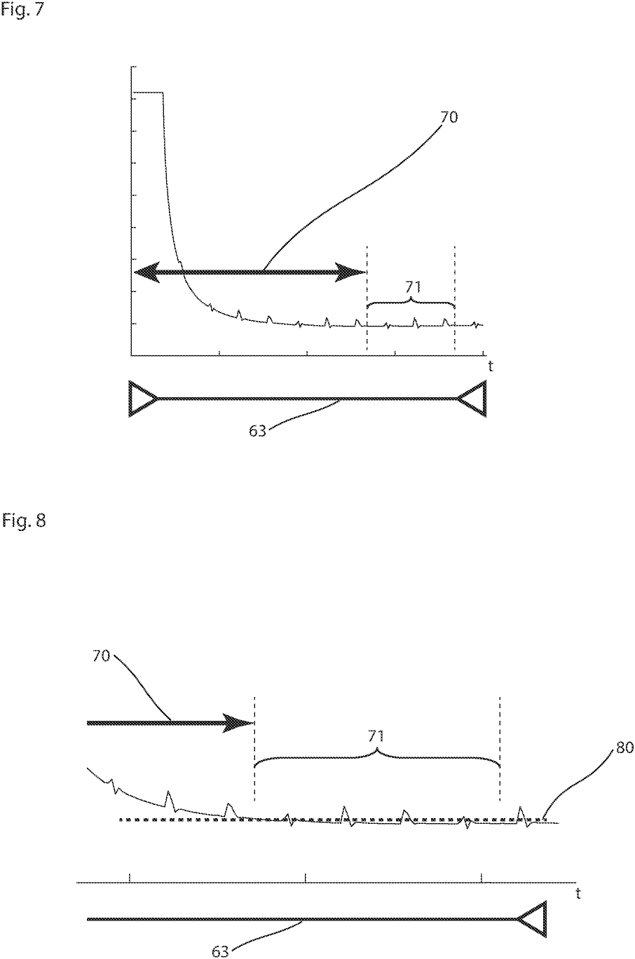

It is common practice in the design of brushless DC and/or stepper motor control to divide the driving time for a specific driving contact into a number of micro-steps, to vary the driving voltage with every micro-step and to spread a pattern of driving voltages over the entire commutation cycle. This means that the driving phases overlap and the driving voltages can be optimized for smoother and more energy efficient commutation. FIG. 6a shows an example of such a driving pattern, with a shape of half a sine wave spread over half of a commutation cycle, illustrated with 16 micro-steps per motor full-step (62) and four motor full-steps (62) per commutation cycle. In this example, the measuring window is positioned at the beginning of the first micro-step of the commutation pattern, and the course of Back EMF voltage signals sampled during the measuring window is shown enlarged. For commutation, the same driving pattern including the same measuring window is applied cyclically to all four driving contacts, as shown in FIG. 6b. In this example, all patterns consist of positive voltages. With every phase having a positive driving contact and a negative driving contact, the voltages on a particular phase could also be seen as a micro-stepped complete sine wave. It is, however, an important aspect of the present disclosure that positive driving contacts and negative driving contacts of all phases are treated separately as shown. The commutation pattern may have any other shape, such as rectangular or trapezoidal, and the measuring window may be positioned anywhere in the commutation pattern for any duration. To perform the measurement with the least possible consequence for commutation, however, it may be advantageous to choose a time for the voltage measurement where no commutation is active, or, in the language of the example in FIGS. 6b, a time where the driving voltage pattern is absent or has zero volt. This is the case whenever the polarity of a phase changes, in other words whenever the control circuitry switches the driving pattern from the positive driving contact of a phase to the negative driving contact of the same phase, or vice versa. In an embodiment with two phases and four driving contacts in the commutation cycle, there will be two such switching events per phase and commutation cycle, which is the same as one switching event per driving pattern of each driving phase. In the example of a micro-stepped half sine wave, there is one micro-step with zero volt, which is either at the beginning or at the end of every pattern, depending on where the start of a cycle is defined to be. In FIG. 6a, the measuring window (63) for the Back EMF voltage signal (60) starts with every driving voltage pattern (61), and every driving voltage pattern (61) starts with one micro-step of zero voltage, which means that the Back EMF measurement can take place without modifying the commutation. In FIG. 6a the duration of the measuring window (63) is shown to be shorter than one micro-step. This is not necessarily always the case, as will be explained further below. FIG. 7 shows a further detailed view of the voltage already enlarged in FIG. 6a, giving an example of a course of voltage measured on the driving contacts of phase A just before beginning to apply a driving voltage pattern, during a measuring window (63) of 0.2 ms with no driving voltage active. The voltage at the driving contacts of phase A in this situation is a combination of voltage induced by switching off coil A (40) and voltage induced in the same coil by the movement of the rotor. As illustrated in FIG. 7, the voltage at or voltage difference between the driving contacts immediately jumps to a high value, due to switching off commutation at the opposite end of the coil (40), then quickly and at least partly exponentially falls down to converge towards a substantially horizontal part of the curve, oscillating around a substantially constant value: the convergence value (80, see FIG. 8). The convergence value is a basis for all subsequent analysis an. As an obvious alternative, instead of measuring the voltage at a driving contact, the electrical current at a driving contact could be measured and analyzed.

In an embodiment of the present disclosure, the convergence value may be determined by sampling, during each measuring window (63), the course of the voltage at a driving contact (41) or the course of the voltage difference between two driving contacts (41) of the paused driving phase, and storing the measured values in a set of convergence data (71), starting at a convergence offset (70) from the beginning of the measuring window (63). Sampling is typically performed by triggering the ADC in the measuring circuitry at a constant sampling rate. The drug delivery device may operate with a sampling rate of at least 1 kHz, such as at least 1 MHz, and the convergence data consist of 1 to 1000, for example 16 convergence data values obtained at a convergence offset of 0 to 1000, such as 184 samples within the measuring window. The duration of the measuring window (63) required to let the initial peak pass and determine the convergence value depends on the inductor time constant Tau of the stator coils, on the driving voltage, on the rotation speed of the motor, on the implementation of the measurement circuitry, and on other factors. No minimum offset can hence be specified for the general method. Choosing a longer measurement window may increase, to a limit, the accuracy of calculating the convergence value, but the disadvantages of pausing the commutation will soon become prohibitive. In a practical realization of the present disclosure, the course of the Back EMF voltage signal will be analyzed for a particular motor, a particular control circuitry and a particular measurement circuitry, and the duration of the measurement window will be adjusted accordingly. As discussed above, an ideal Back EMF voltage signal measurement is designed with as little as possible impact on commutation. However, in many cases the duration of the measuring window required for Back EMF voltage analysis may exceed the duration of a micro-step, at least under certain operating conditions, for example at high speed or if the driving voltage pattern is applied with finely pitched micro-steps. To run the blockage detection, the measuring window will typically get priority over commutation at least on a regular selection of commutations, for example for all applications of a driving voltage pattern. This is illustrated in FIG. 6b, where the measuring windows (63) are visibly longer than even several micro-steps.

To arrive at a single convergence value per measuring window, the voltage measurement circuitry will sample the course of the Back EMF voltage signal at the driving contacts of a driving phase over at least part of the measuring window and evaluate a multitude of measured voltages. In an embodiment of the blockage detection according to the present disclosure, a set of convergence data (71), a selection of sampled voltage values starting at a convergence offset (70) after start of the measuring window (63), is used to determine the convergence value (80) of that particular measuring window. In the example of FIG. 7, the last 16 values sampled in a measuring window are selected as a set of convergence data and used to calculate the convergence value. This process is illustrated in FIG. 8. The convergence value of the measuring window may be calculated as an average or arithmetic mean of all convergence values in the set of convergence data, although it could also be any other mathematical method to get a value representative of the measuring window, such as applying a digital filter, a statistical analysis or any kind of selection. Step 3 of the blockage detection, in all these cases, includes establishing at least one convergence value per measuring window.

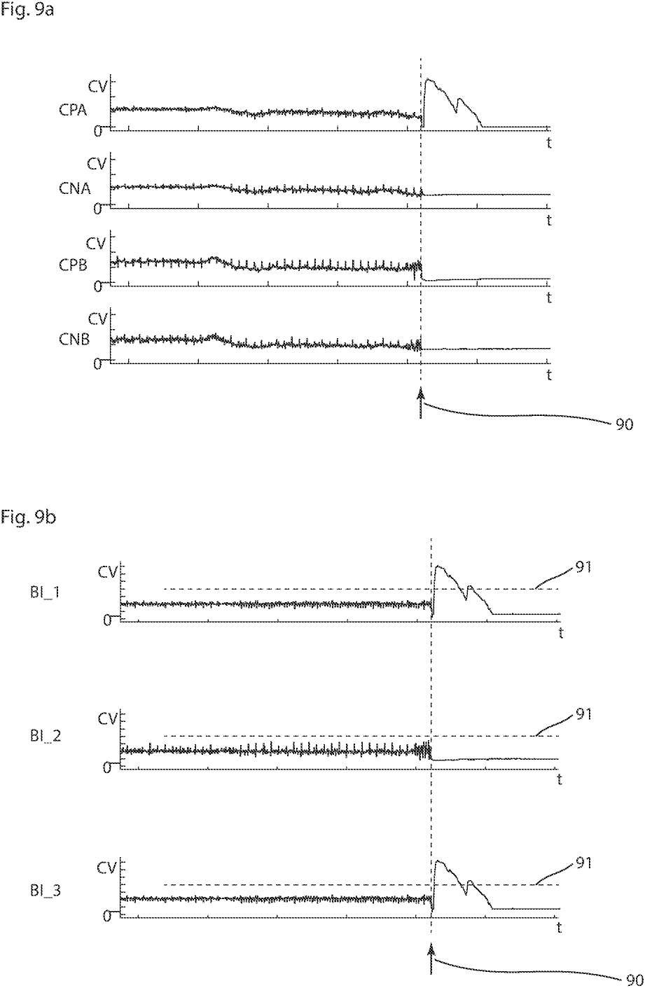

In step 4 of the blockage detection, the divergence of convergence values assigned to different driving contacts is quantified by calculating a blockage index BI. To specify a mathematical expression for a specific embodiment of the blockage detection, it may be helpful to assign each convergence value acquired in steps 1 to 3 to one of the driving contacts. As the Back EMF voltage at the contacts of a coil changes polarity when a driving voltage is switched off, and as the convergence values may be calculated from positive voltages, the most natural assignment is to assign the convergence values derived from voltage measurements on a positive driving contact to the negative driving contact of the same phase, and the convergence values derived from voltage measurements on a negative driving contact to the positive driving contact of the same phase. This is the logic in a typical embodiment of the present disclosure. Other assignment rules could be used, for example to the same driving contact as the measurement was taken from, provided that values from different driving contacts are consistently kept separate from each other and the assignment allows the analysis of the course of convergence values over a multitude of commutation cycles. The assignment of convergence values to a driving contact may be realized by storing convergence values in separate storage elements so that further processing steps can access the values selectively. The blockage detection according to the present disclosure may include the use of multiple convergence values assigned to the same driving contact over a plurality of commutation cycles. Consequently, the assigning step may include storing sequences of convergence values, for example one sequence per driving contact. In the example of an embodiment with two phases A and B, four driving contacts P_{A}, N_{A}, P_{B}, N_{B} and one measurement window per application of a driving pattern, there are two sequences CP_{A}, CP_{B}, each with a sequence of convergence values assigned to a positive driving contact P_{A}, P_{B}, and two sequences CN_{A} CN_{B}, each with a sequence of convergence values assigned to a negative driving contact N_{A}, N_{B}. Every driving contact is measured once per commutation cycle, hence said sequences typically contain one convergence value per commutation cycle. The term “sequence”, in the context of convergence values, refers to the fact that one particular set of data is updated for a succession of commutation cycles. The number of convergence values stored in a storage element holding a sequence depends on the implementation of the method (160) as described in the present disclosure. FIG. 9a shows the sequences CP_{A}, CN_{A}, CP_{B} and CN_{B} in a graphical representation over a time of several minutes. The arrow (90) marks the time of blockage of the actuating assembly, in the example of an embodiment the time of blockage of the plunger. It is clearly visible that the sequence CP_{A} shows a significantly different course of convergence values after blockage than the sequence CN_{A}. The reason for this effect is that, in an actuating system with a minimum (non zero) elasticity in the actuating assembly, the rotor does not immediately get stuck and motionless in case of a blockage. The rotor would move forward until the maximum torque is reached, then fall back a small angle when the commutation recedes. This angular motion of the rotor causes a Back EMF signal. When in this situation the commutation is continued and a commutation pattern is applied to the opposite end of the same phase, the Back EMF signal will be different, typically close to zero, because the rotor will not be in the correct angular position to be advanced by a positive commutation. The rotor may even be reversed one step. Different scenarios of rotor movement at maximum torque in the presence of elasticity of the actuating assembly can be visualized using the schematic illustration of the magnetic structure of the motor in FIG. 4a, FIG. 4b and FIG. 4c. They all lead to a divergence of Back EMF signals measured at opposite ends of the same phase, consequently to a divergence of convergence values assigned to both the positive and the negative driving contact of the same phase, and further consequently to a divergence of convergence values assigned to any selection of different driving contacts, provided said selection contains the positive and the negative driving contact of at least one phase.

As a main part of step 4, the blockage index is derived from the convergence values. The blockage index may be obtained explicitly by calculating a mathematical expression including or corresponding to a subtraction of values assigned to different driving contacts or groups of driving contacts, or implicitly by applying a statistical analysis or comparison of maximum and minimum values over all driving contacts. FIG. 9b shows the graphs of three basic examples of the blockage index BI in an embodiment with two phases A and B:

$$\text{BI\_1} \left(\text{k}\right) = {\text{CP}}_{\text{A}} \left(\text{k}\right) {\text{- CN}}_{\text{A}} \left(\text{k}\right) \text{or}$$

$$\text{BI\_2} \left(\text{k}\right) = {\text{CP}}_{\text{B}} \left(\text{k}\right) {\text{- CN}}_{\text{B}} \left(\text{k}\right) \text{or}$$

$$\text{BI\_3} \left(\text{k}\right) = \text{maximum} \left({\text{CP}}_{\text{A}} \left(\text{k}\right) , {\text{CN}}_{\text{A}} \left(\text{k}\right) , {\text{CP}}_{\text{B}} \left(\text{k}\right) , {\text{CN}}_{\text{B}} \left(\text{k}\right)\right)$$

minimum (CP_{A} (k), CN_{A} (k), CP_{B} (k), CN_{B} (k)) if k is a running number of commutation cycles.

As the divergence of convergence values is especially surprising when observed between convergence values assigned to opposite ends of the same phase, the selection of driving contacts included in calculating the blockage index will typically include both ends of every phase selected, such as both ends of all phases used to drive the motor. Calculating a blockage indication representing the divergence of positive and negative convergence values is not limited to mathematical expressions involving the difference operator, but could be any other means of expressing the difference between the sequences, for example assigning ranges of values, using statistical analysis or applying thresholds, just to name a few. Calculating a blockage indication may also use a combination of explicit and implicit differences, for example by calculating the difference between an actual convergence value and a statistical value like a mean convergence value. Just like the sequences of convergence values, the blockage indication may also be stored sequentially in a storage element of sufficient length to allow further processing, realizing another sliding window of at least one, for example at least ten commutation cycles.

As step 5 of blockage detection, the drug delivery device uses a blockage indication and a blockage criterion to decide if a blockage of the actuation assembly is present. Again, numerous ways exist to specify a criterion, and this criterion obviously depends on how the blockage indication is calculated. Using the example of a simple difference of convergence values on a single phase, exceeding a threshold could be used as an equally simple blockage criterion:

Blockage = YES if BI_1(k) > ±threshold (91) with threshold in the range of, for example, 3 to 6, if using the convergence values as shown in FIG. 9b.

A more robust blockage detection uses multiple blockage indicators calculated at different times, for example by waiting with a blockage decision until threshold (91) has been exceeded multiple times, for example at least three times in the last ten commutation cycles. Similar to the calculation of the blockage indication, the blockage criterion is not limited to a mathematical expression, but could be any other means of taking a decision based on the blockage indication calculated according the method (160) of the present disclosure, for example by using ranges of values, using statistical analysis or applying variable thresholds, just to name a few.

Once the drug delivery device has reached the decision that a blockage of the actuating assembly is present, the device will forward this information to the patient or to another external component involved in controlling the drug delivery. This is step 6 of the blockage detection and will typically include an alarming action to attract external attention. In a simple embodiment, the alarming is done by changing the status of the drug delivery device, and by a control element reading the status and reacting on the change. Other alarming actions could be a visual or acoustic alarm on the drug delivery device itself, or to communicate the blockage to an external device for further control of the reaction, for example to display a warning on a remote control device or smartphone. Alternatively or additionally, the drug delivery device may react to the blockage with a mitigation action, such as stopping the commutation or reversing the commutation to unblock the actuating assembly, or with providing guidance to the user to effectuate further action.

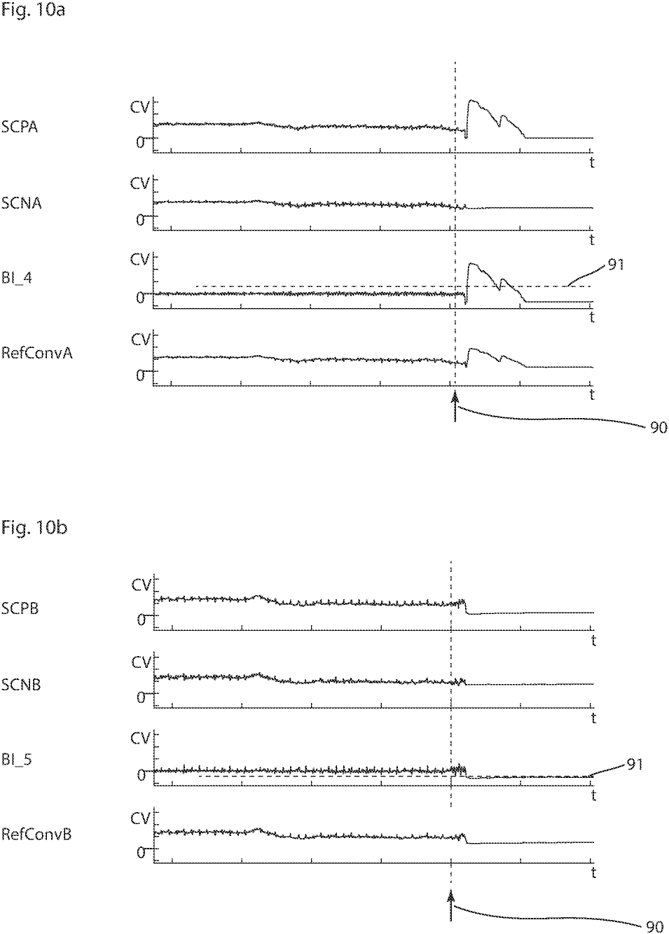

Starting from a drug delivery device with system for blockage detection as described with steps 1 to 6, a number of further improvements can be included. A first further aspect of the present disclosure relates to smoothing of the convergence values. Looking at FIG. 9a, it is apparent that convergence values, even if they are already a result of averaging of a multitude of convergence data values, may show ripples, small oscillations or similar variations when comparing convergence values of subsequent commutation cycles. As a basis for a detection of blockage it may be advantageous to use convergence values with less short-term variation to enhance the contrast to a longer-term change. In a further embodiment of the current disclosure, the convergence values may therefore be smoothed over a time of more than one, for example at least 16 commutation cycles, by applying a digital filter and/or calculating a sliding arithmetic mean, a sliding weighted mean, a sliding median or other mathematical method of smoothing a series of values. The resulting graph of smoothed convergence values looks smoother in short-term variation with a pronounced change when the actuating assembly is blocked. This is illustrated in FIG. 10a (phase A) and FIG. 10b (phase B), where SCP_{A}, SCN_{A}, SCP_{B} and SCN_{B} show smoothed variants of the sequences SCP_{A}, SCN_{A}, SCP_{B} and SCN_{B}. The calculation of a blockage index may remain the same as with original convergence values. Still in FIG. 10, further examples of blockage indices are shown as
BI_4(k) = SCP_{A} (k) - SCN_{A} (k) or
BI_5(k) = SCP_{B} (k) - SCN_{B} (k) with k still as a running number of commutation cycles.
A second further aspect of the present disclosure relates to normalization of convergence values. As the blockage detection is based on differences, and differences can always be expressed as absolute values or else as relative deviations from a reference, another embodiment of the present disclosure may include calculating a sliding reference value RefConv, and expressing all convergence values as relative deviations from said reference RefConv. In such an embodiment, there may be one reference per phase. A more global reference could include RevConv being calculated globally from all convergence values measured during a series of commutation cycles. A simple example of generating a sliding reference is to calculate, for each phase and commutation cycle, the arithmetic mean of the convergence value assigned to the positive driving contact and the convergence value assigned to the negative driving contact. FIG. 10a and FIG. 10b contain graphs for RefConv_{A} and RefConv_{B} which are the sequences of the sliding average of SCP_{A} and SCN_{A}, and of SCP_{B} and SCN_{B} respectively. Normalization is typically performed by dividing each convergence value by the reference value for the same commutation cycle, as for example in the formula

$${\text{N\_SCP}}_{\text{A}} \left(\text{k}\right) = {\text{SCP}}_{\text{A}} \left(\text{k}\right) / {\text{RefConv}}_{\text{A}}$$

with a running number of commutation cycles k, and N_SCP_{A}(k) being a normalized form of the smoothed convergence value SCP_{A}. Normalized convergence values my further be scaled, for example to represent a percentage, as in

$${\text{N\_SCP}}_{\text{A}} \left(\text{k}\right) = {\text{SCP}}_{\text{A}} \left(\text{k}\right) / {\text{RefConv}}_{\text{A}} * \text{100}$$

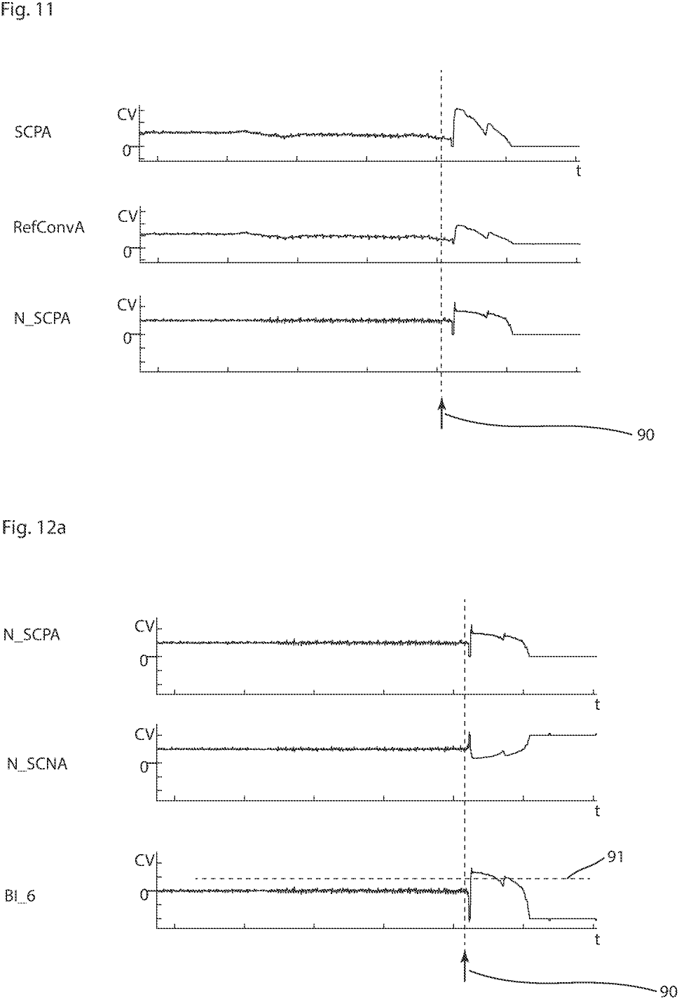

FIG. 11 illustrates the example of normalization of the sequence SCP_{A} as just described.

To avoid division by zero when calculating a normalized convergence value, a small non-zero value may be added to the reference RevConv. If an embodiment of the blockage detection includes smoothing of convergence values, the normalization may take place either before or after performing the smoothing, the latter further offering the advantage of easier implementation. The steps of smoothing a series of convergence values, calculating a reference and normalizing the convergence values could also be performed in one step for optimum efficiency in implementation. Just like unprocessed or smoothed convergence values, normalized convergence values may be used to calculate a blockage indication value based on differences of values assigned to positive driving contacts and values assigned to negative driving contacts. FIG. 12b (bottom) shows a graph of the following example:

$$\text{BI\_6} \left(\text{k}\right) = {\text{N\_SCP}}_{\text{A}} \left(\text{k}\right) {\text{- N\_SCN}}_{\text{A}} \left(\text{k}\right)$$

$$\text{BI\_7} \left(\text{k}\right) = {\text{N\_SCP}}_{\text{B}} \left(\text{k}\right) {\text{- N\_SCN}}_{\text{B}} \left(\text{k}\right)$$

with k as a running number of commutation cycles

As seen when discussing FIG. 9, the difference between convergence values assigned to positive driving contacts and convergence values assigned to negative driving contacts typically appears in one driving phase only. In a system with more than one driving phase, the angular position the rotor in a status of blockage determines which driving phase is showing the difference between convergence values assigned to positive and negative driving contacts. Hence, a third further aspect of the present disclosure relates to calculating the total blockage indication value based on a combination of blockage indices individually calculated for different phases. This extension ensures a reliable blockage detection in a system with more than one driving phase. A simple example of such a combination is

$$\text{BI\_8} \left(\text{k, total}\right) = \text{maximum} \left(\text{BI\_4} \left(\text{k}\right) , \text{BI\_5} \left(\text{k}\right)\right) \text{or}$$

$$\begin{matrix}\text{BI\_8} \left(\text{k}\right) = \text{maximum} \left({\text{SCP}}_{\text{A}} \left(\text{k}\right) {\text{- SCN}}_{\text{A}} \left(\text{k}\right) ,\right) \\ \left({\text{SCP}}_{\text{B}} \left(\text{k}\right) {\text{- SCN}}_{\text{B}} \left(\text{k}\right)\right)\end{matrix}$$

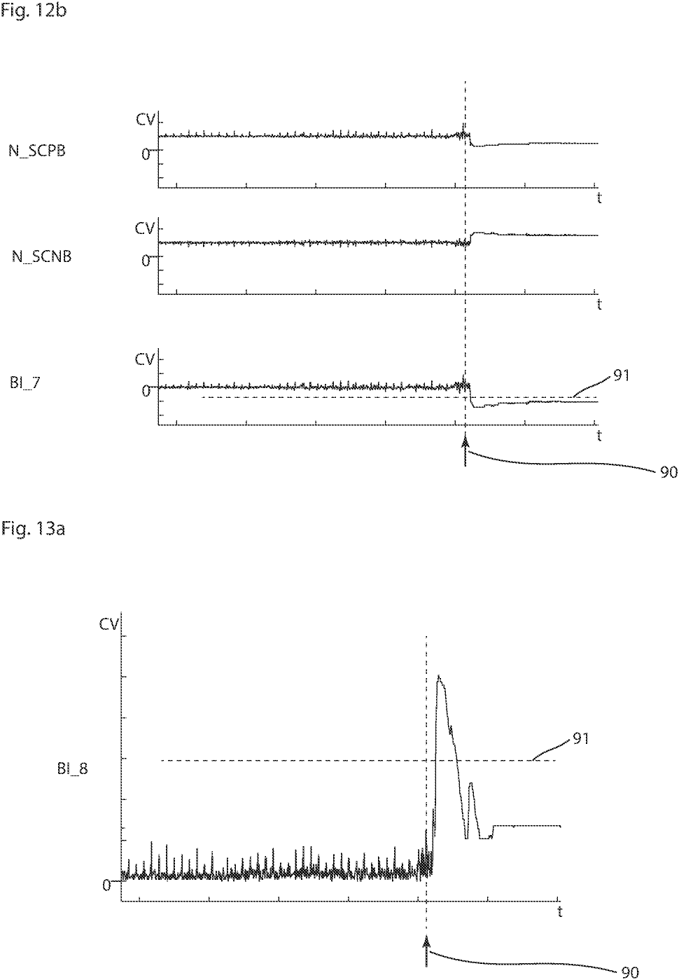

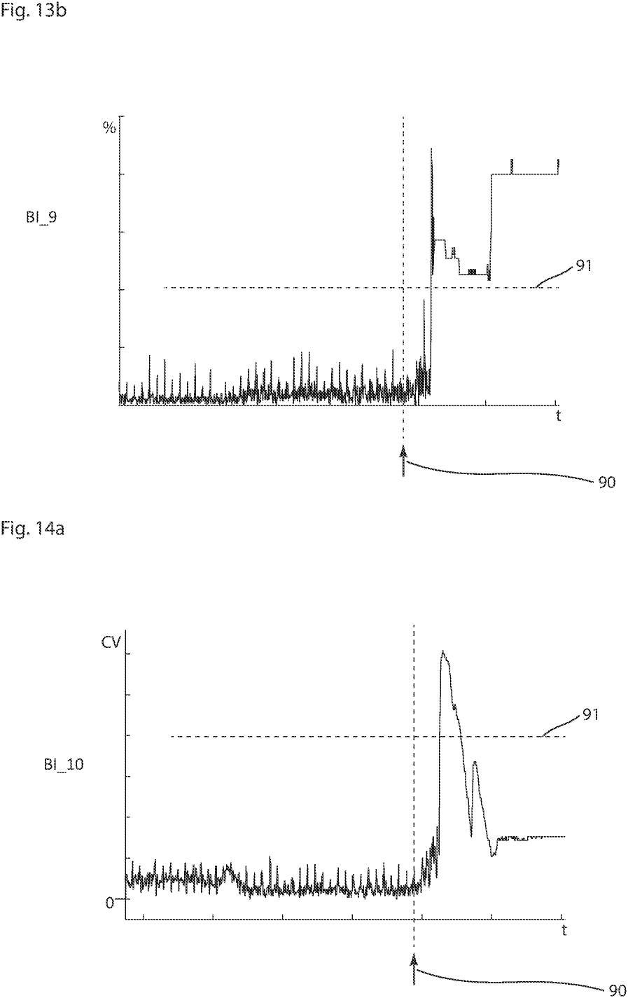

again with k as a running number of commutation cycles, see FIG. 13a. The same combination of phases may of course be calculated from normalized convergence values, as shown in FIG. 13b and expressed in the formula

$$\begin{matrix}\text{BI\_9} \left(\text{k}\right) = \text{maximum} \left({\text{N\_SCP}}_{\text{A}} \left(\text{k}\right) {\text{- N\_SCN}}_{\text{A}} \left(\text{k}\right) ,\right) \\ \left({\text{N\_SCP}}_{\text{B}} \left(\text{k}\right) {\text{- N\_SCN}}_{\text{B}} \left(\text{k}\right)\right)\end{matrix}$$

A fourth further aspect of the present disclosure relates to calculating the blockage indication value based on a combination of convergence values from a multitude of phases rather than from pre-calculated blockage indices from individual phases. The divergence of convergence values may be differences between values assigned to both ends of the same phase, but the same divergences may be visible, and quantifiable, by analyzing the convergence values as assigned to any suitable combination of the driving contacts. One example of such a blockage indication value has already been mentioned as BI_1(k), looking for the maximum variation of values over all convergence values as assigned to any driving contacts. This aspect of the present disclosure includes all sorts of statistical analysis or method for comparison in a set of values. Further simple examples of blockage indication values based on unsorted combinations of driving contacts are variations of BI_1 using sequences of smoothed convergence values SCP, SCN or sequences of normalized convergence values N_SCP, N_SCN:

$$\begin{matrix}\text{BI\_10} \left(\text{k}\right) = \text{maximum} \left({\text{SCP}}_{\text{A}} \left(\text{k}\right) , {\text{SCN}}_{\text{A}} \left(\text{k}\right) , {\text{SCP}}_{\text{B}} \left(\text{k}\right) , {\text{SCN}}_{\text{B}} \left(\text{k}\right)\right) \\ \text{- minimum} \left({\text{SCP}}_{\text{A}} \left(\text{k}\right) , {\text{SCN}}_{\text{A}} \left(\text{k}\right) , {\text{SCP}}_{\text{B}} \left(\text{k}\right) , {\text{SCN}}_{\text{B}} \left(\text{k}\right)\right) \text{and}\end{matrix}$$

$$\begin{matrix}\text{BI\_11} \left(\text{k}\right) = \text{maximum} \left({\text{N\_SCP}}_{\text{A}} \left(\text{k}\right) , {\text{N\_SCN}}_{\text{A}} \left(\text{k}\right) , {\text{N\_SCP}}_{\text{B}} \left(\text{k}\right) ,\right) \\ \left({\text{N\_SCN}}_{\text{B}} \left(\text{k}\right)\right) \text{- minimum} \left({\text{N\_SCP}}_{\text{A}} \left(\text{k}\right) , {\text{N\_SCN}}_{\text{A}} \left(\text{k}\right) ,\right) \\ \left({\text{N\_SCP}}_{\text{B}} \left(\text{k}\right) , {\text{N\_SCN}}_{\text{B}} \left(\text{k}\right)\right) .\end{matrix}$$

as shown in FIG. 14a and FIG. 14b.

Changing the optic from looking at absolute differences to looking at relative deviations or errors, a fifth further aspect of the present disclosure emerges, relating to calculating the blockage indication value as an error or deviation from a normalization reference as described before. Just like selecting convergence values from multiple phases and combining them to a global blocking indication value, the same concept may be applied to normalization. The reference used for normalization may be calculated from multiple phases, stored from earlier commutation cycles, programmed as a fixed constant or obtained in another way to serve the purpose of indicating a deviation of convergence values assigned to positive driving contacts from convergence values assigned to a negative driving contact. When indicating a comparison with a reference, the blockage indication value may be interpreted as an indicator of an error of convergence values. Again, to give a simple example, a blockage indication value may be calculated as the maximum of normalized convergence values for all driving contacts:

$$\begin{matrix}\text{BI\_12} \left(\text{k}\right) = \text{maximum} \left({\text{N\_SCP}}_{\text{A}} \left(\text{k}\right) , {\text{N\_SCN}}_{\text{A}} \left(\text{k}\right) , {\text{N\_SCP}}_{\text{B}} \left(\text{k}\right) ,\right) \\ \left({\text{N\_SCN}}_{\text{B}} \left(\text{k}\right)\right)\end{matrix}$$

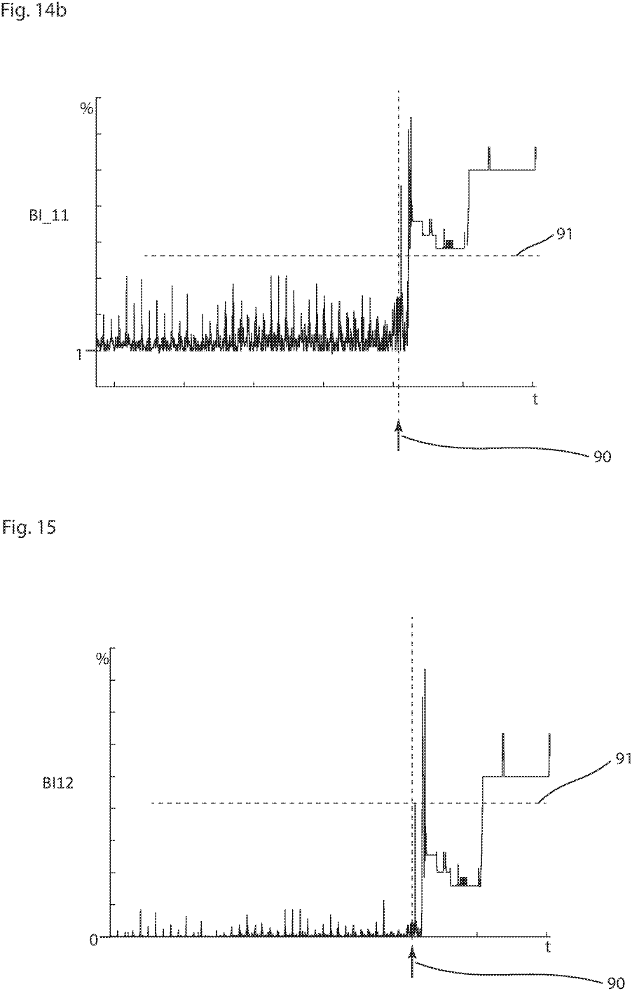

However, in BI_12, a large negative error value would be ignored. A further embodiment of a blockage indication may hence get rid of the leading sign of the convergence value and use the concept of a mean square error. FIG. 15 shows a graph of a blockage indication value with the formula

$$\begin{matrix}\text{BI\_13} \left(\text{k}\right) = \text{maximum} \left({\left({\text{N\_SCP}}_{\text{A}} \left(\text{k}\right)\right)}^{2} , {\left({\text{N\_SCN}}_{\text{A}} \left(\text{k}\right)\right)}^{2} ,\right) \\ \left({\left({\text{N\_SCP}}_{\text{B}} \left(\text{k}\right)\right)}^{2} , {\left({\text{N\_SCN}}_{\text{B}} \left(\text{k}\right)\right)}^{2}\right) .\end{matrix}$$

Obviously, using the absolute value of error values would lead to a similar result.

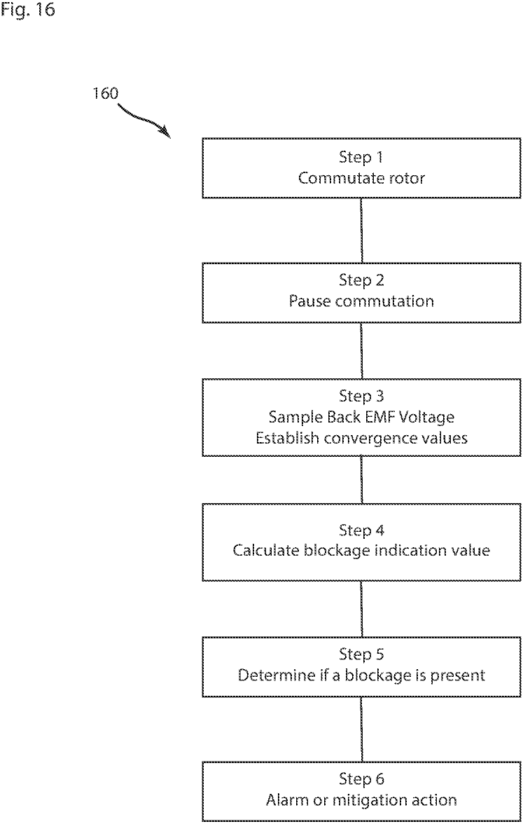

FIG. 16 summarizes steps 1 to 6 of the method (160) for detecting blockage according to the present invention in a flow-chart.

While the description of possible embodiments of the present disclosure in this document has focused on keeping the calculation of the blockage indication values as simple as possible, all sorts of more complicated embodiments may be used with the same effect. The blockage indication value may, for example, use processed or unprocessed convergence values from different commutation cycles rather just one, patterns of values or characteristics of curves may be analyzed, longer term behavior analysis could be included or variation over time like using a non-constant sensitivity for blockage detection. Calculation steps like smoothing, filtering or averaging may be applied in any order or combination. Common to all these variations is that the blockage detection is based on an analysis of differences in Back EMF voltages observed on positive driving contacts and Back EMF voltages observed on negative driving contacts. It is evident that “differences” may not explicitly be expressed as a subtraction in a formula, but could implicitly be included in another mathematical concept like average, mean, correlation, drop, ratio, variance, just to name a few.

While many embodiments have been explained using the example of a wearable patch injector as shown in FIG. 1, it is evident that the current disclosure may also be used in all sorts of pumps for the infusion of drugs like insulin, in handheld electronic injectors, or in any other kind of medical device, wherever a stepper or brushless DC motor is used to effectuate fluid delivery or actuate a mechanical movement which is relevant for a therapy and where hence a reliable blocking detection may be advantageous.

While the invention of disclosure has been described in detail in the drawings and foregoing description, such description is to be considered illustrative or exemplary and not restrictive. Variations to the disclosed embodiments can be understood and effected by those skilled in the art and practising the claimed invention, from a study of the drawings, the disclosure, and the appended claims. In the claims, the word “comprising” does not exclude other elements or steps, and the indefinite article “a” or “an” does not exclude a plurality. The mere fact that certain elements or steps are recited in distinct claims does not indicate that a combination of these elements or steps cannot be used to advantage, specifically, in addition to the actual claim dependency, any further meaningful claim combination shall be considered disclosed.

LIST OF REFERENCE NUMERALS

| | |
|---|---|
| 1 | Patch pump / fluid delivery system |
| 10 | Housing |
| 11 | Command button |
| 12 | Reservoir window |
| 13 | Adhesive patch assembly |
| 20 | Electronic control unit |
| 21 | Reservoir |
| 22 | Plunger |
| 30 | Plunger moving assembly / Actuating assembly |
| 30 a | Gear box assembly |
| 30 b | Threaded rod |
| 30 c | Segmented rod |
| 30 d | Plunger moving head |
| 31 | Stepper or BLDC motor |
| 32 | Fluid transport assembly |
| 32 a | Input |
| 32 b | Output |
| 40 | Coil |
| 40 a | Positive end |
| 40 b | Negative end |
| 41 | Positive driving contact |
| 42 | Negative driving contact |
| 43 | Positive magnetic flux conductor |
| 44 | Negative magnetic flux conductor |
| 45 | Rotor |
| 50 | Control circuitry |
| 51 | MOS-FET switch |
| 55 | Voltage measurement circuitry |
| 60 | Back EMF voltage signal |
| 61 | Driving voltage pattern |
| 62 | Motor full-step |
| 63 | Measuring window |
| 70 | Convergence offset |
| 71 | Convergence data |
| 80 | Convergence value |
| 90 | Time of blockage |
| 91 | Threshold |
| 160 | Method for detecting blockade |

What is claimed is:

1. A method to detect and report blockage of an actuating assembly in a mobile or wearable drug delivery device, wherein the drug delivery device comprises:
an electric stepper motor or brushless DC motor operatively connected to the actuating assembly and comprising at least one driving phase, each driving phase having a coil, a positive driving contact, and a negative driving contact to drive the motor;
a voltage measurement circuitry to measure the voltage at any driving contact or between any pair of driving contacts;
an electronic control unit with a control circuitry, an electrical power source, a microprocessor, memory and software configured to control the commutation of the motor, to supervise the actuation of the drug delivery device and to initiate an alarm and/or a mitigation action if a blockage of the actuating assembly has been detected;
wherein the method comprises
commutating the motor by applying a pattern of positive driving voltages to the driving contacts, in which a commutation cycle is repeated with a succession of all positive driving contacts and all negative driving contacts;
pausing driving of at least one driving phase twice per commutation cycle, by pausing between driving the positive and driving the negative driving contact of the same driving phase, to open a recurring measuring window during which the driving contacts of the paused driving phase are controlled by the voltage measuring circuitry to measure a Back EMF voltage signal at or between said driving contacts;
measuring, for a sequence of measuring windows, the voltage at or between driving contacts of a driving phase and establishing, for each of the sequence of measuring windows, a convergence value representing the Back EMF voltage during the measuring window;
periodically calculating, at least once for every commutation cycle k, a blockage indication value BI based on a divergence of convergence values measured at or between different driving contacts;
determining that a blockage of the actuation assembly is present if the blockage indication value BI exceeds a blockage threshold MaxConvDiff for a specified minimum number of commutation cycles k within a specified minimum blockage duration; and
initiating an alarm and/or a mitigation action if a blockage has been determined.

2. The method according to claim 1, wherein the measuring further comprises:
sampling, during each measuring window, a course of the voltage at a driving contact or the course of the voltage difference between two driving contacts of the paused driving phase, and storing the measured values in a set of convergence data, starting at a convergence offset from the beginning of the measuring window; and
calculating from each set of stored convergence data one convergence value by applying a first digital filter and/or calculating a first arithmetic mean, a first weighted mean or a first median of the values in the set of convergence data.

3. The method according to claim 2, wherein the calculating further comprises:
assigning each convergence value to the positive or to the negative driving contact of the driving phase paused to perform the voltage measurement, such that the convergence value is assigned to a driving contact to which a driving pattern has last been applied before starting the measuring window the convergence value is representing; and
storing the convergence values in sequences per driving contact by storing the convergence values in sequences CP_{A}, CP_{B} for convergence values at the positive driving contacts P_{A}, P_{B} and in sequences CN_{A}, CN_{B} for convergence values at the negative driving contacts N_{A}, N_{B} in a system with two driving phases A and B.

4. The method according to claim 3, wherein the calculating includes calculating the blockage indication value BI for every commutation cycle k based on a difference between convergence values CP assigned to a positive driving contact and convergence values CN assigned to a negative driving contact using the formula
$$\text{BI} \left(\text{k}\right) = {\text{CP}}_{\text{A}} {\text{A - CN}}_{\text{A}} \left(\text{k}\right) \text{using values from driving phase A or}$$
$$\text{BI} \left(\text{k}\right) = {\text{CP}}_{\text{B}} \left(\text{k}\right) - {\text{CN}}_{\text{B}} \left(\text{k}\right) \text{using values from driving phase B} \text{.}$$
.

5. The method according to claim 3, wherein the calculating includes
calculating, for each sequence of convergence values, a sequence of smoothed convergence values SCP_{A}, SCP_{B}, SCN_{A} and SCN_{B}, wherein the sequence of convergence values has been smoothed by applying a second digital filter and/or calculating a sliding second arithmetic mean, a sliding second weighted mean or a sliding second median of at least 16 values in each sequence CP_{A}, CP_{B}, CN_{A} and CN_{B}; and
calculating the blockage indication value BI for every commutation cycle k based on the difference between smoothed convergence values at a positive driving contact SCP_{A} or SCP_{B} and a smoothed convergence value at a negative driving contact SCN_{A} or SCN_{B} using the formula
$$\text{BI} \left(\text{k}\right) = {\text{SCP}}_{\text{A}} \left(\text{k}\right) {\text{-SCN}}_{\text{A}} \left(\text{k}\right) \text{or}$$
$$\text{BI} \left(\text{k}\right) = {\text{SCP}}_{\text{B}} \left(\text{k}\right) {\text{-SCN}}_{\text{B}} \left(\text{k}\right) .$$
.

6. The method according to claim 4, further comprising normalizing the convergence values before using them to calculate the blockage indication value BI; wherein
normalization is performed by mathematically converting all convergence values to a representation of a relative deviation from a sliding reference Ref_{A}, Ref_{B} calculated for each driving phase;
the reference Ref_{A}, Ref_{B} is calculated as a sliding average, weighted mean, arithmetic mean or median of the positive convergence value CP_{A}, CP_{B} and the negative convergence value CN_{A}, CN_{B} of each commutation cycle k;
each conversion value in CP_{A}, CN_{A}, CP_{B} and CN_{B} is normalized by calculating the percentual deviation from Ref_{A} or Ref_{B} using the formula Norm_CPₓ(k) = (CPₓ(k) / Ref_{y}(k)) * 100 with placeholders x = A or B and y = A or B.

7. The method according to claim 5, further comprising the step of normalizing the smoothed convergence values before using them to calculate the blockage indication value BI; whereby
normalization is performed by mathematically converting all smoothed convergence values to a representation of a relative deviation from a sliding reference Ref_{A}, Ref_{B} calculated for each driving phase;
the reference Ref_{A}, Ref_{B} is calculated as a sliding average, weighted mean, arithmetic mean or median of the smoothed positive convergence value SCP_{A}, SCP_{B} and the smoothed negative convergence value SCN_{A}, SCN_{B} of each commutation cycle k;
each smoothed conversion value in SCP_{A}, SCN_{A}, SCP_{B} and SCN_{B} is normalized by calculating the percentual deviation from Ref_{A} or Ref_{B} using the formula Norm_SCPₓ(k) = (SCPₓ(k) / Ref_{y}(k)) * 100 with placeholders x = A or B and y = A or B.

8. The method according to claim 5, wherein the blockage indication value is based on a combination of blockage indication values calculated for a plurality of individual driving phases, using the formula:
$$\begin{matrix}\text{BI} = max \left({\text{SCP}}_{\text{A}} - {\text{SCN}}_{\text{A;}} {\text{SCP}}_{\text{B}} - {\text{SCN}}_{\text{B}}\right) > \\ \text{MaxConvDiff using the smoothed convergence values} \text{.}\end{matrix}$$
.

9. The method according to claim 7, wherein the blockage indication value is based on a combination of blockage indication values calculated for a plurality of individual driving phases, using the formula:
$$\begin{matrix}\text{BI} = max \\ \left({\text{Norm\_SCP}}_{\text{A}} {\text{- Norm\_SCN}}_{\text{A;}} {\text{Norm\_SCP}}_{\text{B}} {\text{- Norm\_SCN}}_{\text{B}}\right) \\ \text{using the normalized smoothed convergence values} \text{.}\end{matrix}$$
.

10. The method according to claim 3, wherein the calculation of the blockage indication value is based on a maximum difference of convergence values in a plurality of driving phases, as for using the formula:
$$\begin{matrix}\text{BI} = max \left({\text{SCP}}_{\text{A}} ; {\text{SCN}}_{\text{A}} \text{;} {\text{SCP}}_{\text{B}} \text{;} {\text{SCN}}_{\text{B}}\right) \text{-} \\ min \left({\text{SCP}}_{\text{A}} ; {\text{SCN}}_{\text{A}} \text{;} {\text{SCP}}_{\text{B}} \text{;} {\text{SCN}}_{\text{B}}\right) .\end{matrix}$$
.

11. The method according to claim 7, wherein the calculation of the blockage indication value is based on a maximum deviation from a normalization reference using the formula:
$$\begin{matrix}\text{BI} = max \\ \left({\text{Norm\_SCP}}_{\text{A}} ; {\text{Norm\_SCN}}_{\text{A}} \text{;} {\text{Norm\_SCP}}_{\text{B}} \text{;} {\text{Norm\_SCN}}_{\text{B}}\right) \text{or}\end{matrix}$$
$$\begin{matrix}\text{BI} = max {\left({\left({\text{Norm\_SCP}}_{\text{A}}\right)}^{2} ; {\left({\text{Norm\_SCN}}_{\text{A}}\right)}^{2} ; \left({\text{Norm\_SCP}}_{\text{B}}\right)\right)}^{2} \\ ; \left({\left({\text{Norm\_SCN}}_{\text{B}}\right)}^{2}\right) .\end{matrix}$$
.

12. The method according to claim 1, wherein the voltage measurement operates with a sampling rate of at least 1 kHz, and the convergence data comprises at least 1 convergence data value obtained at a convergence offset of 0 to 1000 samples within the measuring window.

13. The method according to claim 1, wherein the voltage measurement operates with a sampling rate of at least 1 MHz, and the convergence data comprises at least 16 convergence data values obtained at a convergence offset of at least 184 samples within the measuring window.

14. A system to detect and report blockage of an actuating assembly in a mobile or wearable drug delivery device, wherein
the drug delivery device comprises an electric stepper motor or brushless DC motor operatively connected to the actuating assembly and comprising at least one driving phase, each driving phase having a coil, a positive driving contact, and a negative driving contact to drive the motor;
the system comprises a voltage measurement circuitry to measure the voltage at or between the driving contacts;
the system comprises an electronic control unit with a control circuitry, an electrical power source, a microprocessor, storage and software configured to control the commutation of the motor, to supervise the actuation of the drug delivery device and to initiate an alarming action if a blockage of the actuating assembly has been detected;
wherein the system detects blockage by:
commutating the motor by applying a pattern of positive driving voltages to the driving contacts, in which a commutation cycle is repeated with a succession of all positive driving contacts and all negative driving contacts;
pausing driving of at least one driving phase at least twice per commutation cycle, by pausing between driving the positive and driving the negative driving contact of the same driving phase, to open a recurring measuring window during which the driving contacts of the paused driving phase are controlled by the voltage measuring circuitry to measure a Back EMF voltage signal at or between said driving contacts;
measuring, for a sequence of measuring windows, the voltage at or between driving contacts of a driving phase and establishing, for each of the sequence of measuring windows, a convergence value representing the Back EMF voltage during said measuring window;
assigning each convergence value to the positive driving contact or to the negative driving contact of the driving phase paused to perform the voltage measurement, such that the convergence value is assigned to a driving contact to which the driving pattern has last been applied before starting the measurement window the convergence value is representing;
periodically calculating, at least once for every commutation cycle k, a blockage indication value BI based on a divergence of convergence values assigned to different driving contacts;
determining that a blockage of the actuation assembly is present if the blockage indication value BI meets or exceeds a blockage threshold MaxConvDiff for a specified minimum number of commutation cycles k within a specified minimum blockage duration.

15. The system according to claim 14, wherein the voltage measurement operates with a sampling rate of at least 1 kHz, and the convergence data consists of at least 1 convergence data value obtained at a convergence offset of 0 to 1000 samples within the measuring window.

16. The system according to claim 14, wherein the voltage measurement operates with a sampling rate of at least 1 MHz, and the convergence data consists of at least 16 convergence data values obtained at a convergence offset of at least 184 samples within the measuring window.

17. The system according to claim 14, wherein the drug delivery device comprises a reservoir for containing a medical fluid, a plunger movably disposed in the reservoir and a fluid transport assembly to connect the reservoir to a patient, and wherein the actuating assembly is a plunger moving assembly to move the plunger in the reservoir and effectuate fluid delivery to the patient.
